(19) United States
(12) Patent Application Publication
(10) Pub. No.: US 2019/0110857 A1
(43) Pub. Date: Apr. 18, 2019

(54) SKIN MARKING TEMPLATE FOR RADIOGRAPHIC IMAGING

(71) Applicant: Christopher Pettis, Orlando, FL (US)

(72) Inventor: Christopher Pettis, Orlando, FL (US)

(21) Appl. No.: 15/787,176

(22) Filed: Oct. 18, 2017

(51) Int. Cl. A61B 90/00; A61M 25/01

(52) U.S. Cl. CPC A61B 90/39; A61M 25/0108

(57) ABSTRACT

A marking template for use during radiographic imaging comprising a body, a first end at one end of the body capable of being used as a handle and a second end, opposite the first end. The second end comprises a radiopaque material and an opening, in relation to the radiopaque material, through which a marking may be made with a marking device.

### FIELD OF INVENTION

The present disclosure generally relates to devices used to determine proper needle placement in the field of radiology; more specifically, to a skin marking template for use during radiographic imaging.

### BACKGROUND

A number of radiographic imaging methods are currently used to visualize internal structures in a body. These imaging methods may be used to help diagnose disease, structural infirmities, and to guide therapeutic procedures. Exemplary radiographic imaging methods include radiography, fluoroscopy, and computerized tomography (CT).

When using these imaging methods to create images of internal structures, anatomical features that are visible on the patient and on the image can be used as points of reference for determining proper location for the entry of invasive devices through the skin, such as needle insertion, or for other surgical or therapeutic procedures. In these cases, a metallic or other radiopaque instrument, such as, for example, a hemostat, paperclip or needle, and a skin marking device, usually in the form of a marker or marking pen, are used to locate and mark a specific location on the skin surface. Since the radiopaque instrument will be visible in the image, the instrument is placed over a patient's body and repositioned during imaging until the radiopaque instrument overlies the desired radiopaque point of reference in the human body, usually a bony structure when using fluoroscopy, and then the marking device is used to mark the location on the patient's skin. Once the procedure is completed, the radiopaque instrument and the marking device are typically discarded. Occasionally, the radiopaque instrument may be re-sterilized at a significant cost and reused. Simply reusing the same radiopaque instrument and pen on multiple patients significantly increases the risk of infection being spread between patients and many hospitals have instituted policies prohibiting such activity.

### SUMMARY

In accordance with the teachings disclosed herein, embodiments related to a skin marking device for radiographic imaging are disclosed.

In an embodiment, the skin marking template comprises a body having a first end and a second opposite end. The first end can include a handle, clip, cap or other apparatus to attach the template to a marking device such as a pen. The second opposite end can include a radiopaque material to allow a user to see the location of the template during a radiographic procedure. The second end can further include a circular shape in which or around which the radiopaque material is disposed to provide the user with a clear view of the location of the template during a radiographic procedure. The template may be placed along a patient's body to locate an area requiring marking by a user. An aperture or channel may be disposed within the second end of the template to allow the user to trace or outline a mark on the patient's body to indicate a specific desired location.

### BRIEF DESCRIPTION OF THE DRAWINGS

FIG. 1 is a perspective view of a skin marking template for radiographic imaging according to aspects of the present invention.

FIG. 2A is a top elevational view of a skin marking template for radiographic imaging according to aspects of the present invention.

FIG. 2B is a side elevational view of a skin marking template for radiographic imaging according to aspects of the present invention.

FIG. 3 is a is a perspective view of an alternate embodiment of a skin marking template for radiographic imaging according to aspects of the present invention.

FIG. 4A is a top plan view of an alternate embodiment of a skin marking template for radiographic imaging according to aspects of the present invention.

FIG. 4B is a side plan view of an alternate embodiment of a skin marking template for radiographic imaging according to aspects of the present invention.

FIG. 5 is a perspective view of an alternate embodiment of a skin marking template for radiographic imaging according to aspects of the present invention.

FIG. 6A is a top plan view of an alternate embodiment of a skin marking template for radiographic imaging according to aspects of the present invention.

FIG. 6B is a side plan view of an alternate embodiment of a skin marking template for radiographic imaging according to aspects of the present invention.

FIG. 7 is a perspective view of an alternate embodiment of a skin marking template for radiographic imaging according to aspects of the present invention.

FIG. 8A is a top plan view of an alternate embodiment of a skin marking template for radiographic imaging according to aspects of the present invention.

FIG. 8B is a side plan view of an alternate embodiment of a skin marking template for radiographic imaging according to aspects of the present invention

### DETAILED DESCRIPTION OF THE PREFERRED EMBODIMENTS

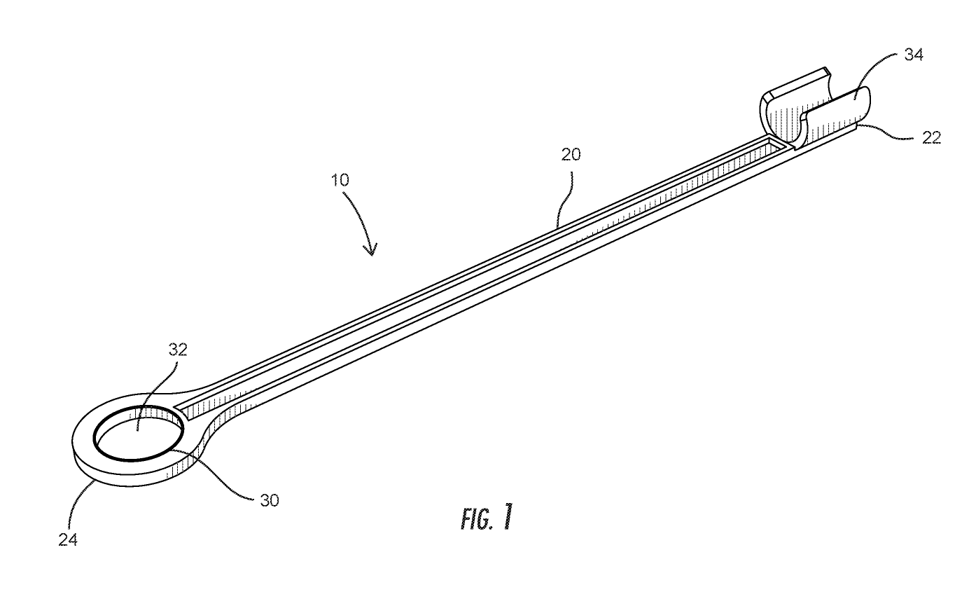

A detailed description of the embodiments for a skin marking device for radiographic imaging will now be presented with reference to FIGS. 1 through 8B. One of skill in the art will recognize that these embodiments are not intended to be limitations on the scope, and that modifications are possible without departing from the spirit thereof. In certain instances, well-known methods, procedures, components, and circuits have not been described in detail.

Embodiments of the present invention can be used as a template to mark a patient's skin during radiographic imaging.

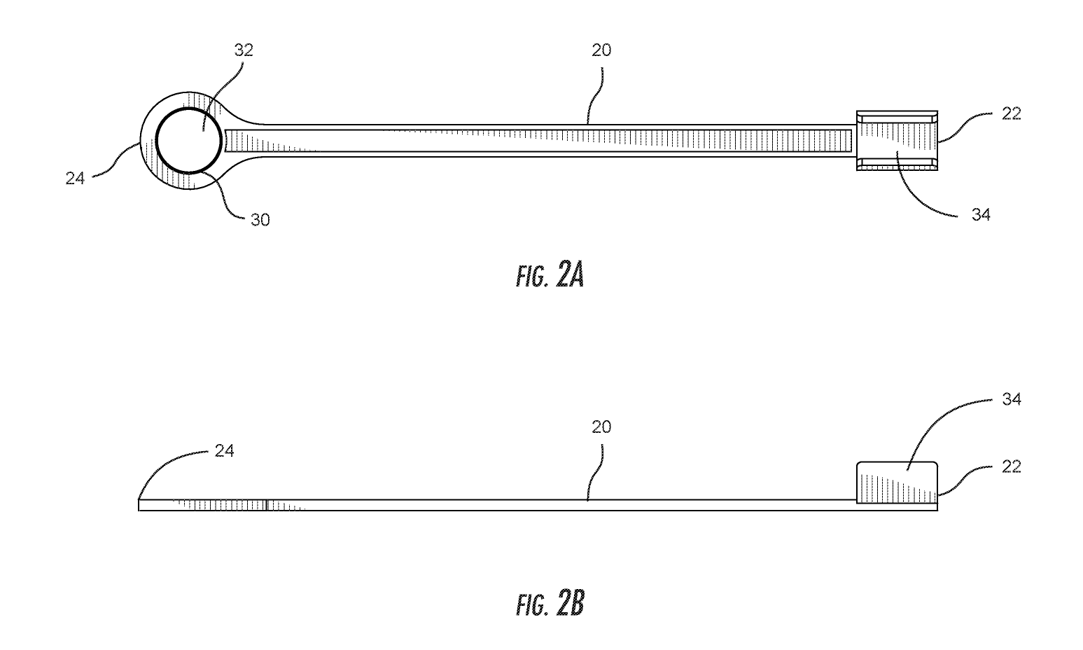

Referring now to FIGS. 1 through 2B, a further alternate embodiment of the template 10 includes a body 20, a first end 22 and a second end 24 opposite the first end 22. The first end 22 may be used as a handle when using the template 10. The second end 24 comprises a radiopaque material 30 and an opening 32 through which a marking device (not shown) may be inserted for marking a patient's skin (not shown). The body 20 may be made from any material that can be sterilized and can comprise a slide or telescopic extension (not shown) to lengthen the template as needed. The first end 22 can include a clip 34 for attachment to a device, such as for example a marking device (not shown). The second end 24 can comprise a generally circular shape. The radiopaque material 30 can be disposed generally about the periphery of the opening 32 in the second end 24 and can be attached to the template 10, embedded in the template 10 or the template 10 may be made all or in part from radiopaque material. The opening 32 in the second end 24 can comprise an open aperture wherein the marking device can be used to trace a mark around the inner portion of the radiographic material 30 disposed about the periphery of the opening 32.

By way of example, and referring back to FIGS. 1 through 2B, the radiopaque material 30 may be disposed about the entire periphery of the opening 32. This is not intended to be limiting as the radiopaque material 30 may be disposed about only a portion of the opening 32, so long as it is sufficient to be seen by a user for proper placement and use.

By further way of example, and again referring back to FIGS. 1 through 2B, the second end 24, as shown, may comprise a circular shape; however, this should not be construed as limiting—second end 24 can be made into a variety of shape and sizes. Furthermore, the opening 32 can be circular in shape, as shown in the drawings; however, this should not be construed as limiting—opening 32 can be made into a variety of shapes and sizes.

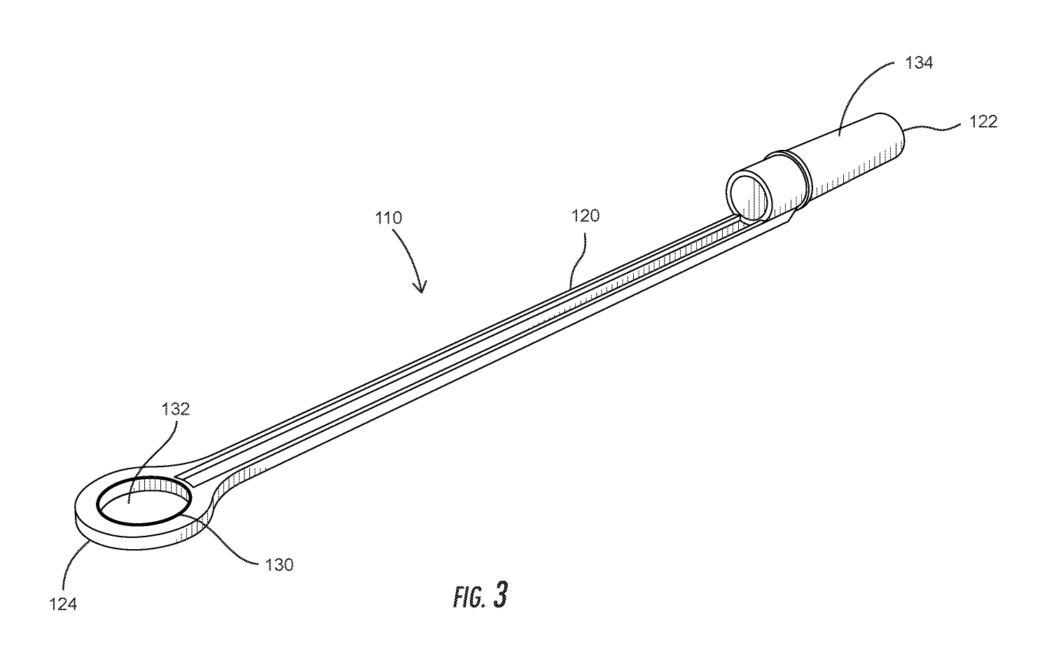

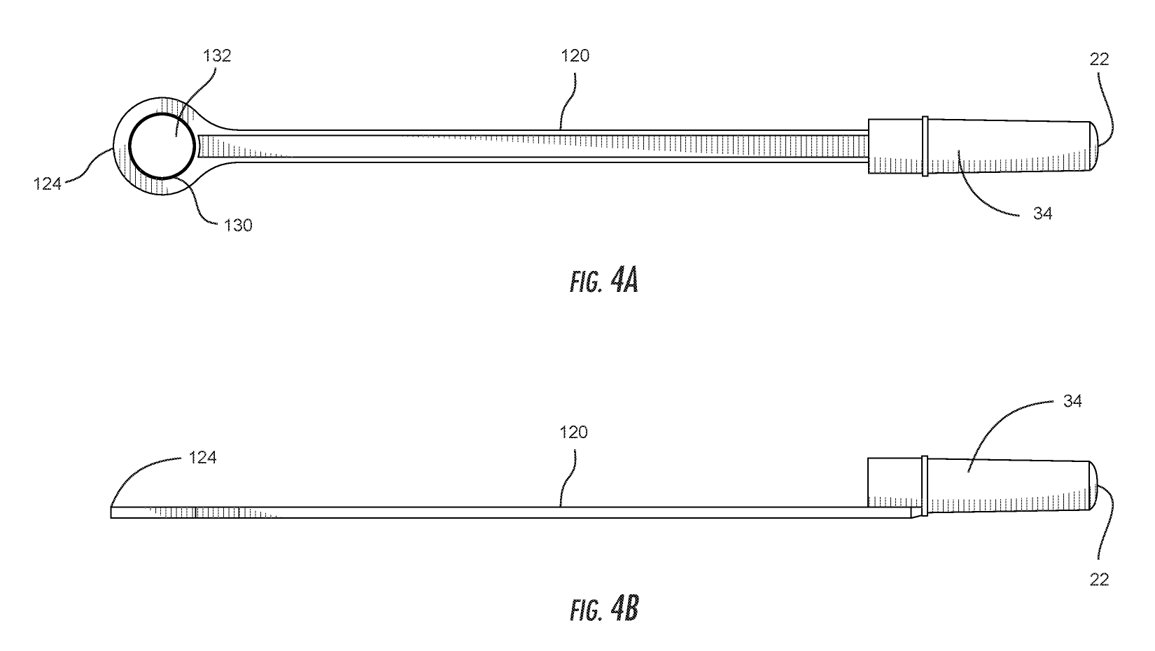

Referring next to FIGS. 3 through 4B, an even further alternate embodiment of the template 110 includes a body 120, a first end 122 and a second end 124 opposite the first end 122. The first end 122 may be used as a handle when using the template 110. The second end 124 comprises a radiopaque material 130 and an opening 132 through which a marking device (not shown) may be inserted for marking a patient's skin (not shown). The body 120 may be made from any material that can be sterilized and can comprise a slide or telescopic extension (not shown) to lengthen the template as needed. The first end 122 can include a cap 134, similar to a marker cap or a pen cap, for attachment to a separate marking device (not shown). The second end 124 can comprise a generally circular shape. The radiopaque material 130 can be disposed generally about the periphery of the opening 132 in the second end 124 and can be attached to the template 110, embedded in the template 110 or the template 110 may be made all or in part from radiopaque material. The opening 132 in the second end 124 can comprise an open aperture wherein the marking device can be used to trace a mark around the inner portion of the radiographic material 130 disposed about the periphery of the opening 132.

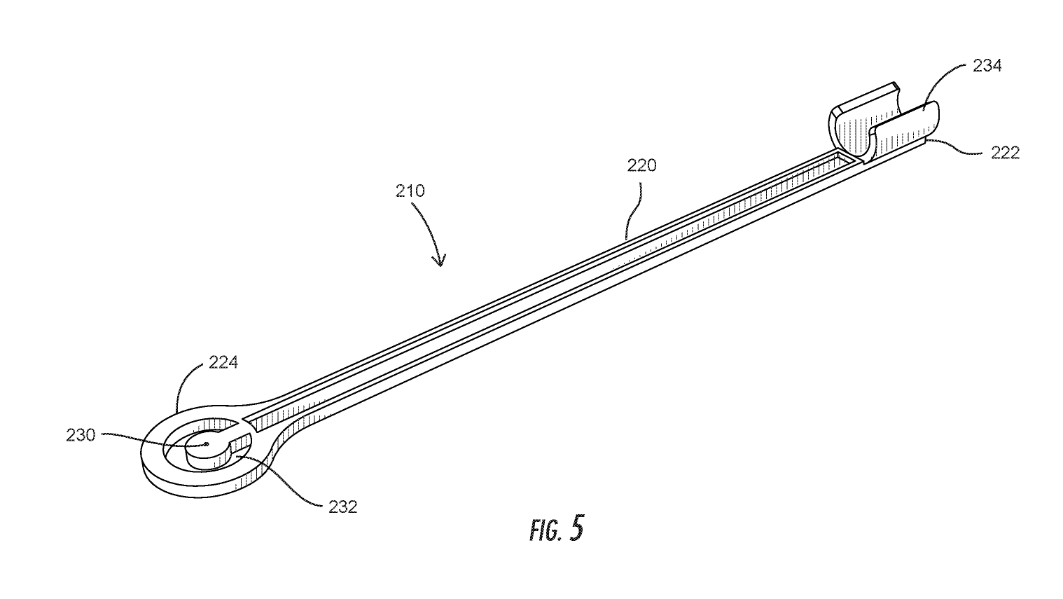

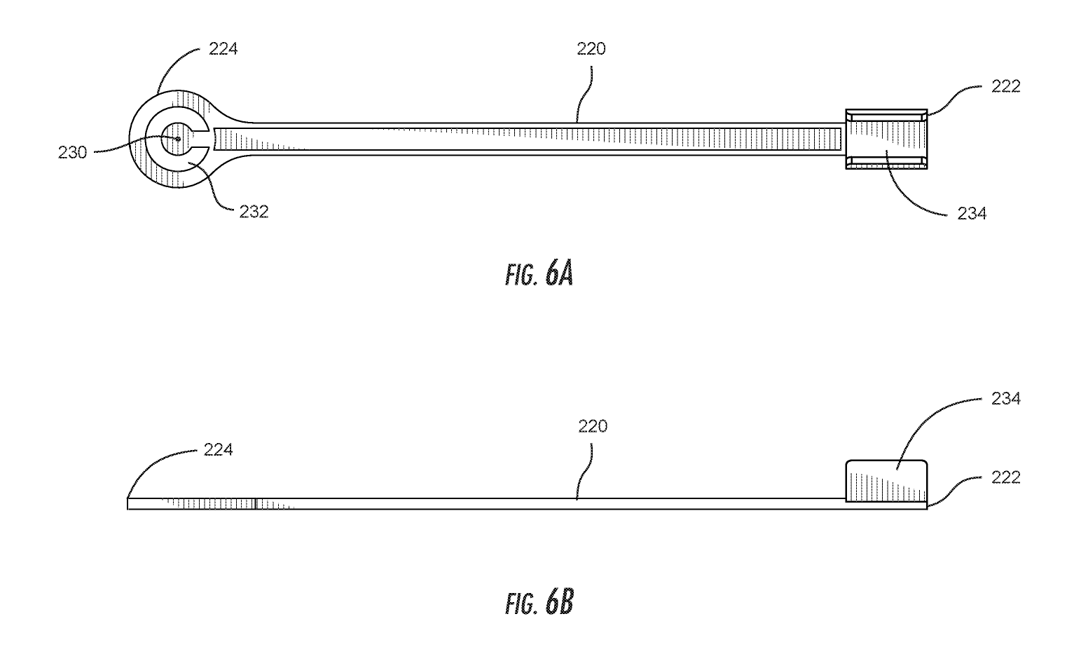

Referring now to FIGS. 5 through 6B, the template 210 includes a body 220, a first end 222 and a second end 224 opposite the first end 222. The first end 222 may be used as a handle when using the template 210. The second end 224 comprises a radiopaque material 230 and an opening 232 through which a marking device (not shown) may be inserted for marking a patient's skin (not shown). The body 220 may be made from any material that can be sterilized and can comprise a slide or telescopic extension (not shown) to lengthen the template as needed. The first end 222 can include a clip 234 for attachment to a device, such as for example a marking device (not shown). The second end 224 can comprise a generally circular shape. The radiopaque material 230 can be disposed generally in the center of the second end 224 and can be attached to the template, embedded in the template or the template may be made all or in part from radiopaque material. The opening 232 in the second end 224 can comprise a channel disposed about the periphery of the radiopaque material 230. The opening 232 can further comprise a substantially semi-circular shape to allow marking around the radiographic material 230.

During a radiographic procedure, the user may view the template 210 under radiographic conditions wherein the radiopaque material 230 can be seen and placed in the desired location for marking. Leaving the template 210 in place, the user may then use a marking device, such as, for example, a marker or a pen (not shown) to mark or to trace the opening 230 thereby creating at least a substantially semi-circular shaped mark around the location of the radiopaque material 230 on the skin of the patient.

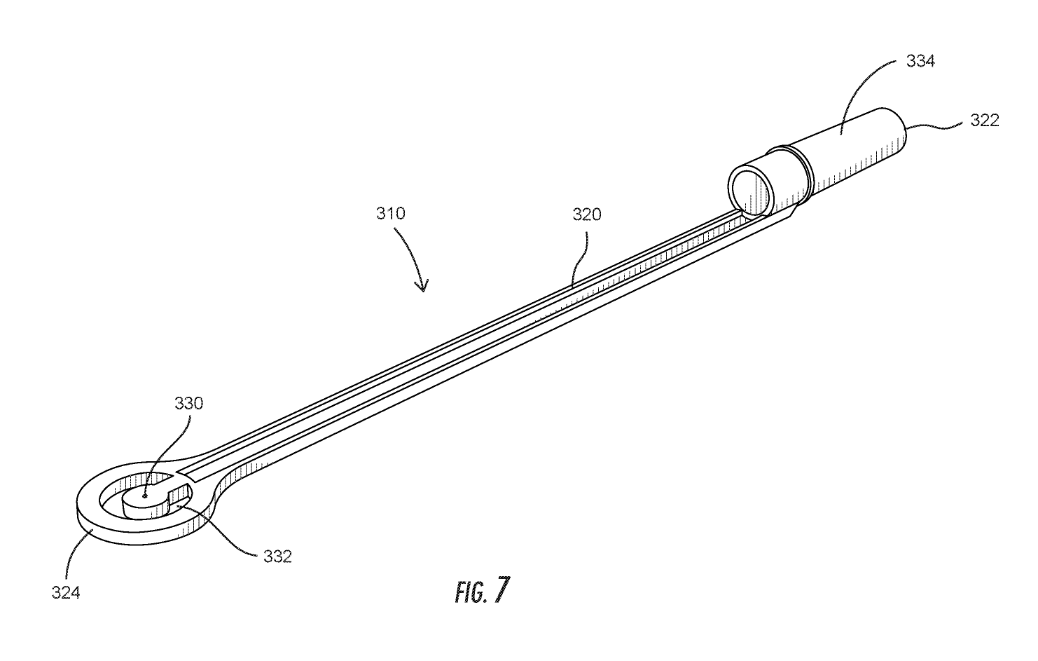

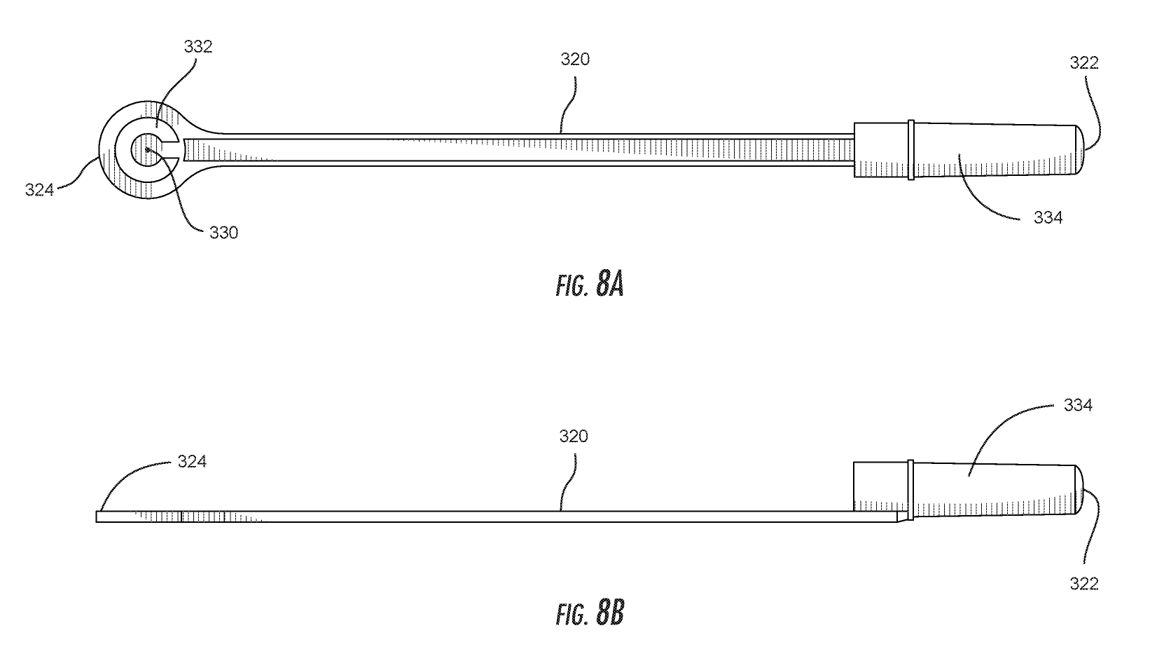

Referring next to FIGS. 7 through 8B, an alternative embodiment is shown. The template 310 includes a body 320, a first end 322 and a second end 324 opposite the first end 322. The first end 322 may be used as a handle when using the template 310. The second end 324 comprises a radiopaque material 330 and an opening 332 through which a marking device (not shown) may be inserted for marking a patient's skin (not shown). The body 320 may be made from any material that can be sterilized and can comprise a slide or telescopic extension (not shown) to lengthen the template as needed. The first end 322 can include a cap 334, similar to a marker cap or a pen cap, for attachment to a separate marking device (not shown). The second end 324 can comprise a generally circular shape. The radiopaque material 330 can be disposed generally in the center of the second end 324 and can be attached to the template, embedded in the template 310 or the template 310 may be made all or in part from radiopaque material. The opening 332 in the second end 324 can comprise a channel disposed about the periphery of the radiopaque material 330. The opening 332 can further comprise a substantially semi-circular shape to allow marking around the radiographic material 330.

As used herein, “radiopaque” includes any substance that at least partially prevents transmission (by blocking, reflecting, absorbing, defracting, and/or any similar phenomena) of at least one type of electromagnetic radiation, such that an image of the substance will be visible in radiographic images. Radiopaque markings using radiopaque materials can be used to assist a user in proper placement of the skin marking device during radiographic imaging. The radiopaque markings used can be of any shape or size and can be made from any known radiopaque material. Exemplary radiopaque materials include titanium, platinum, tungsten, barium sulfate, zirconium oxide, bismuth subcarbonate, stainless steel and iodine.

Having now described the invention, the construction, the operation and use of preferred embodiments thereof, and the advantageous new and useful results obtained thereby, the new and useful constructions, and reasonable mechanical equivalents thereof obvious to those skilled in the art, are set forth in the appended claims.

What is claimed is:

1. A skin marking template comprising:
a body;
a first end at one end of the body capable of being used as a handle; and
a second end, opposite said first end, comprising a radiopaque material and an opening through which a marking may be made with a separate marking device.

2. The skin marking template of claim 1, wherein the second end comprises a generally circular shape.

3. The skin marking template of claim 2, wherein the opening comprises a substantially circular aperture.

4. The skin marking template of claim 3, wherein the radiopaque material is disposed about the periphery of the substantially circular aperture.

5. The skin marking template of claim 3, wherein the radiopaque material is disposed about a portion of the periphery of the substantially circular aperture.

6. The skin marking template of claim 2, wherein the radiopaque material is generally disposed in the center of the circular second end.

7. The skin marking template of claim 6, wherein the opening comprises a channel disposed about the periphery of the radiopaque material.

8. The skin marking template of claim 7, wherein said channel is substantially semi-circular.

9. The skin marking template of claim 1, wherein said first end further comprises a clip.

10. The skin marking template of claim 1, wherein said first end further comprises a cap.

11. The skin marking template of claim 10, wherein said cap is complementary to said separate marking device.

12. The skin marking template of claim 1, wherein said body is extendable.

13. The skin marking template of claim 12, wherein said body is telescopically extendable.

14. A marking template for use during radiographic imaging comprising:
a body;
a first end at one end of the body capable of being used as a handle; and
a second end, opposite said first end, comprising a radiopaque material and an opening, in relation to said radiopaque material, through which a marking may be made with a separate marking device.

15. The skin marking template of claim 14, wherein the radiopaque material is disposed about the periphery of the opening.

16. The skin marking template of claim 14, wherein the radiopaque material is disposed about a portion of the periphery of the opening.

17. The skin marking template of claim 14, wherein the radiopaque material is generally disposed in the center of the second end.

18. The skin marking template of claim 17, wherein the opening comprises a channel disposed about the periphery of the radiopaque material.

19. The skin marking template of claim 14, wherein said first end further comprises a cap.

20. A skin marking template comprising:
a body;
a first end at one end of the body capable of being used as a handle; and
a second end, opposite said first end, comprising an aperture further comprising a radiopaque material disposed about at least a portion of the periphery of the aperture to identify the location of the template under radiologic conditions, and wherein a user may use a separate marking device to make a mark on a patient's skin within the aperture.
